(19) United States
(12) Patent Application Publication
(10) Pub. No.: US 2026/0215706 A1
(43) Pub. Date: Jul. 30, 2026

(54) OXYGEN SATURATION MEASUREMENTS

(71) Applicant: Meta Platforms Technologies, LLC, Menlo Park, CA (US)

(72) Inventors: Aashna Hemkumar, San Jose, CA (US); Xiyu Duan, SAMMAMISH, WA (US)

(21) Appl. No.: 19/038,968

(22) Filed: Jan. 28, 2025

(51) Int. Cl. A61B 5/1455; A61B 5/00

(52) U.S. Cl. CPC A61B 5/14552; A61B 5/681; A61B 5/7225

(57) ABSTRACT

A wearable device includes a light source, a light sensor, and processing logic. The light source is configured to emit illumination light. The light sensor configured to generate light measurements of returning light. The processing logic is configured to generate the light measurements with the light sensor to produce oxygen saturation measurements. The processing logic is also configured to, when the light measurements are below a light threshold, increase a sampling rate of subsequent light measurements by the light sensor and reduce an analog offset of the subsequent light measurements.

### TECHNICAL FIELD

This disclosure relates generally to wearables, and in particular to oxygen saturation monitoring.

### BACKGROUND INFORMATION

Sensors for measuring oxygen saturation are included in wearables, such as smartwatches and fitness trackers. Some wearables utilize photoplethysmography (PPG) technology to measure oxygen saturation. PPG sensors emit light through the skin and detect changes in blood flow, allowing for heart rate monitoring. PPG sensors also emit light through the skin and detect the amount of oxygen in the bloodstream. Oxygenated and deoxygenated blood are different in color and have different absorption levels at different wavelengths and this is used to calculate oxygen saturation. Slight changes in the wavelength of the light may influence the accuracy of the oxygen saturation measurements. These sensors can provide valuable insights and allow users to track their physical activity, stress levels, overall well-being, and/or other metrics.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified.

FIG. 1 illustrates a PPG system that includes a wearable including an oxygen saturation measurement system measuring oxygen level in the blood, in accordance with aspects of the disclosure.

FIG. 2 illustrates an example wearable that may include oxygen saturation monitoring systems, in accordance with aspects of the disclosure.

FIG. 3 illustrates an example circuit block diagram for a transmit path for driving a light source to emit illumination light, in accordance with aspects of the disclosure.

FIG. 4 illustrates an example circuit block diagram of a receive path for generating light measurements, in accordance with aspects of the disclosure.

FIG. 5 illustrates a flow chart of an example process of improving oxygen saturation monitoring accuracy, in accordance with aspects of the disclosure.

FIG. 6 illustrates a flow chart of an example process of oxygen saturation sampling based on a skin perfusion value, in accordance with aspects of the disclosure.

### DETAILED DESCRIPTION

Embodiments of improving oxygen saturation accuracy are described herein. In the following description, numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In aspects of this disclosure, visible light may be defined as having a wavelength range of approximately 380 nm-700 nm. Non-visible light may be defined as light having wavelengths that are outside the visible light range, such as ultraviolet light and infrared light. Infrared light having a wavelength range of approximately 700 nm-1 mm includes near-infrared light. In aspects of this disclosure, near-infrared light may be defined as having a wavelength range of approximately 700nm-1.6 μm.

In aspects of this disclosure, the term “transparent” may be defined as having greater than 90% transmission of light. In some aspects, the term “transparent” may be defined as a material having greater than 90% transmission of visible light.

Sensing techniques for oxygen saturation in blood are generally quite accurate. One of those techniques includes PPG sensing. However, there are contexts where PPG oxygen saturation sensing techniques have sub-optimal performance. One particular context where PPG oxygen saturation sensing techniques may have sub-optimal performance is when a wearable having a PPG sensing system is worn by an individual with darker skin tones, having lower perfusion. This is because the return photodiode current of a PPG signal is more highly attenuated for users with higher melanin concentrations corresponding to low perfusion tissue. As such, the return photodiode signal may have low direct current (DC) and alternating current (AC) signals. Existing technologies attempt to compensate for this by overdriving the PPG sensing by increasing the drive current of the light source (e.g. LED). However, this results in significant power consumption and reduced battery life for some users. In addition, increasing the drive current of an LED results in a wavelength shift of the LED. For example, the wavelength of the LED may shift approximately 2 nm which translates into meaningful error in the oxygen saturation measurements.

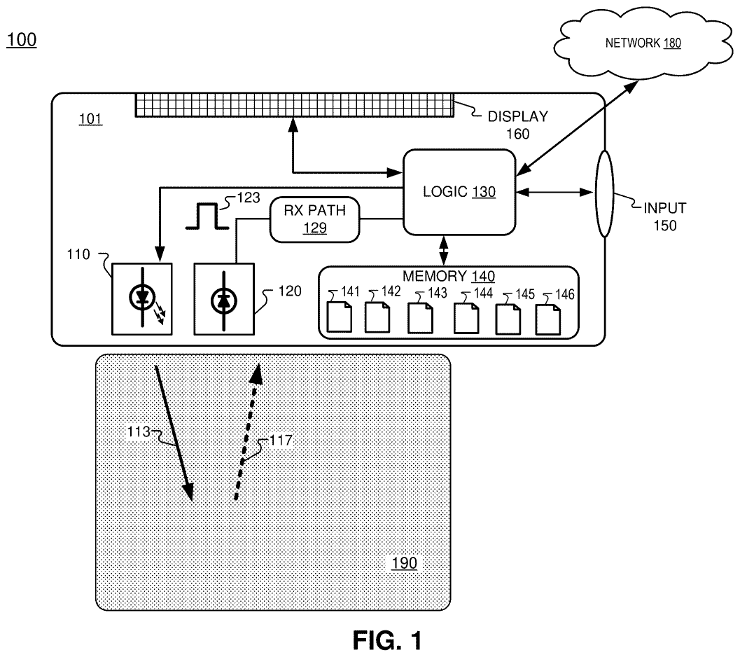

In implementations of the disclosure, light measurements are generated with a light sensor of a wearable to produce oxygen saturation measurements. When the light measurement is below a light threshold, a sampling rate of subsequent light measurements is increased and an analog offset of the subsequent light measurements is reduced. Light measurements below a light threshold may indicate a wearable is being worn by a user with darker skin tones. Instead of increasing the brightness of the light source (e.g. increasing current through an illumination LED), the sampling rate of future light measurements can be increased. Increasing the sampling rate rather than increasing the current through the LED assists in preventing wavelength-shift in the light source that contributes to errors in the measurement, while maintaining the signal to noise ratio (SNR) required for SpO₂. Hence, the same current through the LED may be used for users with different skin tones, in some implementations. In addition to increasing the sampling rate of the subsequent light measurements, an analog offset of subsequent light measurements may also be reduced. The analog offset may be needed for light measurements above the light threshold in order to extend a dynamic range of the light measurements. However, when the light measurements are below the light threshold, the analog offset may be reduced while still having sufficient dynamic range. These and other embodiments are described in more detail in connection with FIGS. 1-6.

FIG. 1 illustrates an oxygen saturation monitoring system 100 that includes a wearable 101 including an oxygen saturation monitoring system measuring oxygen level in blood, in accordance with aspects of the disclosure. Oxygen saturation monitoring system 100 may be a PPG sensing system.

The example wearable 101 illustrated in FIG. 1 includes input(s) 150. Input 150 may include buttons, dials, and/or touch-sensitive sensors, for example. Input(s) 150 are communicatively coupled to processing logic 130, in FIG. 1. In some implementations, inputs for wearable 101 are received via a touch-screen overlaying display 160. Display 160 may be a liquid crystal display (LCD) or an organic light-emitting-diode (OLED) display, for example. Display 160 is also communicatively coupled to processing logic 130, in FIG. 1.

In operation, light source 110 emits illumination light 113 into tissue 190. Light source 110 may include an LED or a laser diode, for example. Light source 110 may emit visible illumination light 113. Illumination light 113 may be red visible light. Light source 110 may be an infrared light source emitting infrared illumination light 113. Light source 110 may be a near-infrared light source emitting near-infrared illumination light 113. For example, light source 110 may be centered around 850 nm or 940 nm.

Illumination light 113 propagates into tissue 190 that includes blood vessels and blood capillaries. A portion of illumination light 113 is reflected/scattered back through tissue 190 and exits tissue 190 as returning light 117 that is measured by light sensor 120. Light sensor 120 may include a photodiode, as illustrated in FIG. 1. In some implementations, light sensor 120 includes an optical filter tuned to receive the wavelength of illumination light 113 (and returning light 117) while blocking out other light wavelengths. In other words, the filter on light sensor 120 may be matched to light source 110.

Light sensor 120 generates signals 123 in response to incident light. When light source is not activated (not emitting illumination light 113), light sensor 120 may generate ambient light measurements as signal 123 to measure the contribution of light from the external environment. When light source 110 is activated (emitting illumination light 113), light sensor 120 generates light measurements of returning light 117 as signal 123. Processing logic 130 may be configured to coordinate driving light source 110 and the sampling of signals 123 by light sensor 120. By generating many light measurements, the heart rate and/or blood oxygen levels of a user of wearable 101 can be determined by measuring corresponding absorption of illumination light 113 in the blood. Other user metrics may also be determined from oxygen saturation measurements. For example, more blood in tissue 190 will absorb more of illumination light 113 and thus returning light 117 will be of a decreased intensity when there is more blood in tissue 190 while returning light 117 will have increased intensity when there is less blood present in tissue 190. Similarly, blood in tissue 190 may be oxygenated or deoxygenated and thus returning light 117 will be modified accordingly. If light 113 is red visible light, returning light 117 will be of a decreased intensity if the blood is deoxygenated compared to when it is oxygenated. If light 113 is infrared illumination light, returning light 117 will be of a decreased intensity if the blood is oxygenated compared to when it is deoxygenated. Patterns can then be extracted from the many light measurements and the patterns can be analyzed for heart rate monitoring or oxygen saturation measurements, for example.

Processing logic 130 may receive signals 123 from light sensor 120 and store the many signals as light measurements in memory 140. Receive path logic 129 may be coupled between light sensor 120 and processing logic 130. Receive path logic 129 may include analog and/or digital circuitry to amplify and/or condition signal 123 for input into processing logic 130. Processing logic 130 may process and analyze light measurements stored in memory 140 to determine oxygen saturation, heart rate, sleep patterns, fitness data, (or otherwise) and then display results to the user via display 160, in some implementations.

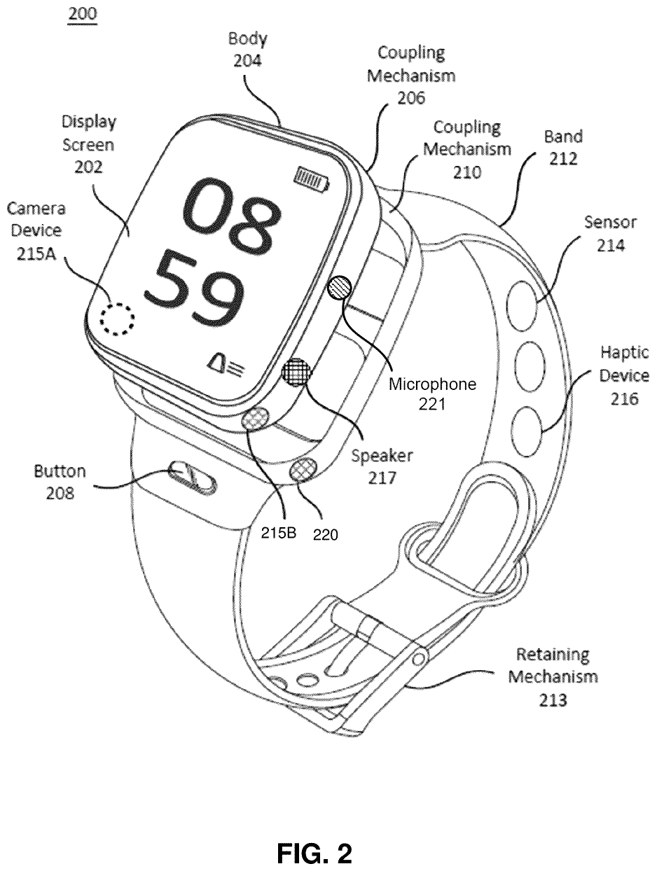

FIG. 2 illustrates an example wearable 200 that may include oxygen saturation monitoring systems such as system 100 of FIG. 1, in accordance with aspects of the disclosure. In some embodiments, a user may select a function by interacting with the button 208 (e.g., by pushing, turning, etc.). In some embodiments, a user may select a function by interacting with the display screen 202. For example, the display screen 202 is a touchscreen and the user may select a particular function by touching the display screen 202, in some implementations. The functions executed by wearable 200 may include, without limitation, displaying visual content to the user (e.g., displaying visual content on the display screen 202), presenting audio content to the user (e.g., presenting audio content via the speaker 217), sensing user input (e.g., sensing a touch of button 208, sensing biometric data with the one or more sensors 214, sensing neuromuscular signals with the one or more sensors 214, etc.), capturing audio content (e.g., capturing audio with microphone 221), capturing data describing a local area (e.g., with a front-facing camera device 215A and/or a rear-facing camera device 215B), communicating wirelessly (e.g., via cellular, near field, Wi-Fi, personal area network, etc.), communicating via wire (e.g., via the port), determining location (e.g., sensing position data with a sensor 214), determining a change in position (e.g., sensing change(s) in position with an IMU), determining an orientation and/or acceleration (e.g., sensing orientation and/or acceleration data with an IMU), providing haptic feedback (e.g., with the haptic device 216), etc.

The display screen 202 may display visual content to the user. The displayed visual content may be oriented to the eye gaze of the user such that the content is easily viewed by the user. Traditional displays on smartwatches may orient the visual content in a static manner such that when a user moves or rotates the smartwatch, the content may remain in the same position relative to the smartwatch causing difficulty for the user to view the content. Embodiments of the present disclosure may orient (e.g., rotate, flip, stretch, etc.) the displayed content such that the displayed content remains in substantially the same orientation relative to the eye gaze of the user (e.g., the direction in which the user is looking). The displayed visual content may also be modified based on the eye gaze of the user. For example, in order to reduce the power consumption of wearable 200, the display screen 202 may dim the brightness of the displayed content, pause the displaying of video content, or power down the display screen 202 when it is determined that the user is not looking at the display screen 202. In some examples, one or more sensors 214 of the wearable 200 may determine an orientation of the display screen 202 relative to an eye gaze direction of the user.

Wearable 200 may be considered a smartwatch. FIG. 2 illustrates a coupling mechanism 206, a camera device 215A, a display screen 202, a button 108, a speaker 217, a microphone 221, and a release mechanism 220 associated with the watch body 204. FIG. 2 illustrates a coupling mechanism 210, a retaining mechanism 213, the sensor 214, the haptic device 216, and a release mechanism 220 associated with the watch band 212. In some implementations, oxygen saturation sensing components (e.g. light source 110 and light sensor 120) are disposed on an underside of the watch body 204. The oxygen saturation sensing components may also be included in band 212, such as within sensor 214, in some implementations.

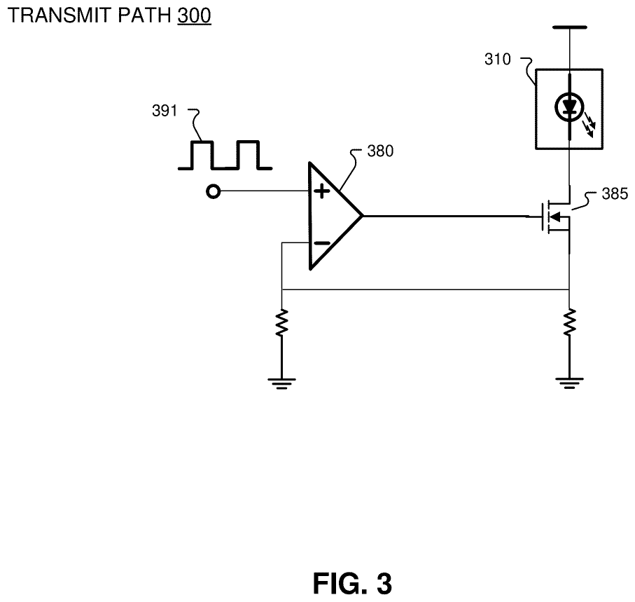

FIG. 3 illustrates an example circuit block diagram for a transmit path 300 for driving a light source 310 to emit illumination light, in accordance with aspects of the disclosure. Transmit path 300 includes an amplifier 380 (e.g. an op-amp) driving a n-channel field-effect transistor (nFET) 385 to control a current through light source 310. Light source 310 may include an LED or a laser diode, for example. Light source 310 may emit visible illumination light. The illumination light may be red visible light. Light source 310 may be an infrared light source emitting infrared illumination light. Light source 310 may be a near-infrared light source emitting near-infrared illumination light. For example, light source 310 may be centered around 850 nm or 940 nm.

In FIG. 3, amplifier 380 receives driving signal 391. Driving signal 391 controls the voltage output of amplifier 380, which modulates the voltage on the gate of nFET 385 to modulate the current through nFET 385, and consequently, light source 310. Driving signal 391 may be driven onto amplifier 380, by processing logic 130, in some implementations. The driving signal 391 may turn light source 310 on and off as well as modulate the intensity of the illumination light emitted from light source 310 while light source 310 is on. In some implementations, increasing the sampling rate of light measurements includes increasing the pulses in driving signal 391 in a given time period (e.g. increasing the pulses per second) to match a particular sampling rate of a light sensor (e.g. light sensor 120). Notably, in some implementations, more than one light source is included in transmit path 300 and each light source may be modulated independently. In these implementations, the transmit path 300 of FIG. 3 may be duplicated and processing logic 130 may control the additional transmit path(s) with separate driving signals.

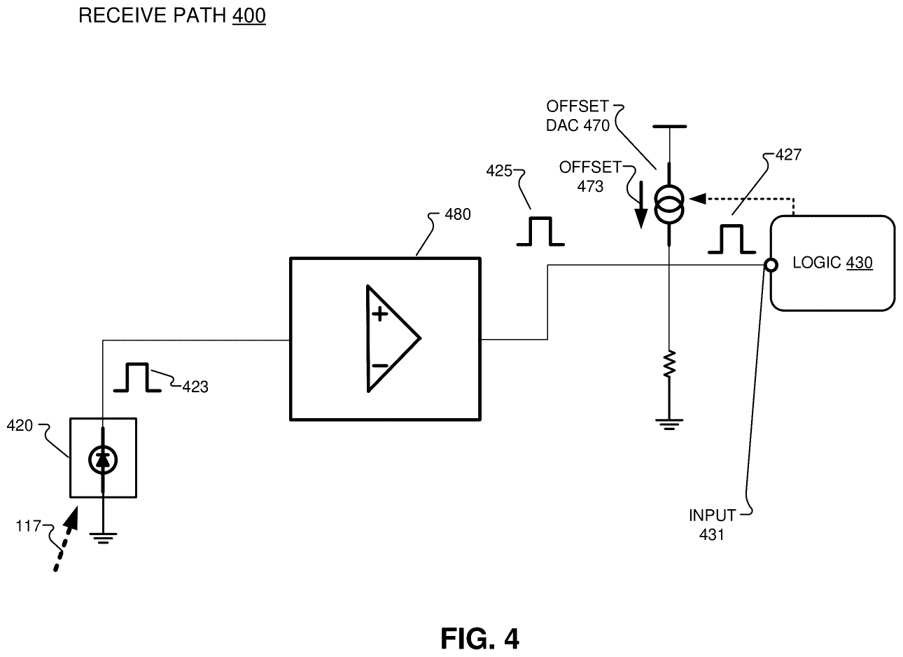

FIG. 4 illustrates an example circuit block diagram of receive path 400 for generating light measurements, in accordance with aspects of the disclosure. Receive path 400 includes light sensor 420, amplifier stage 480, offset digital-to-analog converter (DAC) 470 and processing logic 430. Receive path 400 may be considered an analog front end (AFE) for processing logic 430. Receive path logic 129 may use aspects of receive path 400.

Light sensor 420 may include a photodiode. In some implementations, light sensor 420 includes an optical filter tuned to receive the wavelength of returning light 117 while blocking out other light wavelengths.

In operation, light sensor 420 generates signal 423 in response to returning light 117 incident on light sensor 420. Signal 423 may be a current signal from a photodiode, in some implementations. Optional amplifier stage 480 may generate amplified signal 425 in response to receiving signal 423. Amplifier stage 480 may include one or more op-amps and/or transistors to amplify signal 423.

Offset DAC 470 is coupled between light sensor 420 and processing logic 430. Processing logic 430 may selectively drive Offset DAC 470 to set the offset current 473. Offset DAC is configured to selectively subtract current from the photodiode current (e.g. signal 423 or 425) to extend the dynamic range of measuring the photodiode current. For example, for returning light 117 with high intensity, the photodiode current is high and offset current 473 reduces the signal (e.g. signal 423 or 425) to within the range of an analog-to-digital converter (ADC) that measures signal 423 or 425. In some implementations, a current source generator is driven by a (DAC) controlled by processing logic 430 in order to provide offset current 473. The DAC may be included in the same chip as processing logic 430 or be external to processing logic 430. Hence, the current source generator may be controlled to adjust signal 423/425 received by input 431 of processing logic 430 by selecting the magnitude of offset current 473. Input 431 of processing logic 430 may be an analog-to-digital converter (ADC) that is internal to processing logic 430 or external to processing logic 430. The features of processing logic 430 may be included in processing logic 130.

Referring again to FIG. 1, processing logic 130 may generate light measurements with light sensor 120. Generating the light measurements may include processing logic 130 driving light source 110/310 to emit illumination light 113 while light sensor 120 measures returning light 117. Processing logic 130 may store the light measurements to memory 140 as light measurements 142.

To assist in measuring oxygen saturation for different skin tones (low perfusion skin tones in particular) when the light measurements are below a light threshold 141, processing logic 130 may: (1) increase a sampling rate of subsequent light measurements; and/or (2) reduce an analog offset of the subsequent light measurements. When light measurements 142 are below the light threshold 141, it may indicate a user with darker skin (e.g. FP5 or FP6 on the Fitzpatrick scale). Processing logic 130 may compare the light threshold 141 (stored in memory 140) and one or more of the light measurements 142 (also stored in memory 140) to determine if the light measurement(s) is lower than light threshold 141. The subsequent light measurements (with increased sampling rate and/or reduced analog offset) may be more accurate than the initial light measurements.

In an implementation, increasing the sampling rate of the subsequent light measurements includes increasing an initial sampling rate to an increased sampling frequency of driving the light source and increasing the measuring of the returning light with the light sensor to the increased sampling frequency. Increasing to the increased sampling frequency may include processing logic 130/430 modifying driving signal 391 while also executing analog-to-digital conversions at an input (e.g. input 431) of processing logic 130 at the increased sampling frequency. The subsequent light measurement(s) may be stored in memory 140 as subsequent light measurements 143.

In an implementation, reducing the analog offset of the subsequent light measurements includes processing logic 430 driving offset DAC 470 to have offset current 471 become zero or close to zero. In an implementation, reducing the analog offset of the subsequent light measurements includes reducing an offset current 473 that offsets a photodiode current (e.g. signal 423) generated by a photodiode during the subsequent light measurements. In an implementation, the offset current 473 is generated by offset DAC 470 driven by processing logic 430. Processing logic 430 is configured to receive an adjusted signal 427 that is a difference between the photodiode current (e.g. signal 423 or signal 425) and the offset current 473. Adjusted signal 427 (with reduced or eliminated analog offset) may be measured as part of the subsequent light measurements 143.

Light source 110/310 may be driven at a same current for generating the light measurements 142 and for generating the subsequent light measurements 143. Driving light source 110/310 at the same current (instead of increasing the intensity of the light source) assists in keeping the wavelength of illumination light 113 from shifting. As a result, the oxygen saturation measurements based on the light measurements 142 (and subsequent light measurements 143) have improved accuracy.

In implementations of the disclosure, when light measurements are not below light threshold 141, processing logic 130/430 is configured to (1) generate the subsequent light measurements 143 at the same sampling rate as the initial light measurements 142 and (2) maintain the analog offset for the subsequent light measurements.

In some implementations, memory 140 includes a skin perfusion value 144. The skin perfusion value 144 may be based on previous light measurements by wearable 101. The skin perfusion value 144 may be inputted by the user as a setting of wearable 101. The skin perfusion value 144 may be generated from a photograph of the user that is stored in memory 140 or that processing logic 130 may access via network 180. In some implementations, a pre-determined value range 145 stored in memory 140 may correspond with skin tone types on the Fitzpatrick scale. In an implementation, pre-determined value range 145 corresponds with FP1, FP2, FP3, and FP4 on the Fitzpatrick scale. FP5 and/or FP6 may be outside pre-determined value range 145.

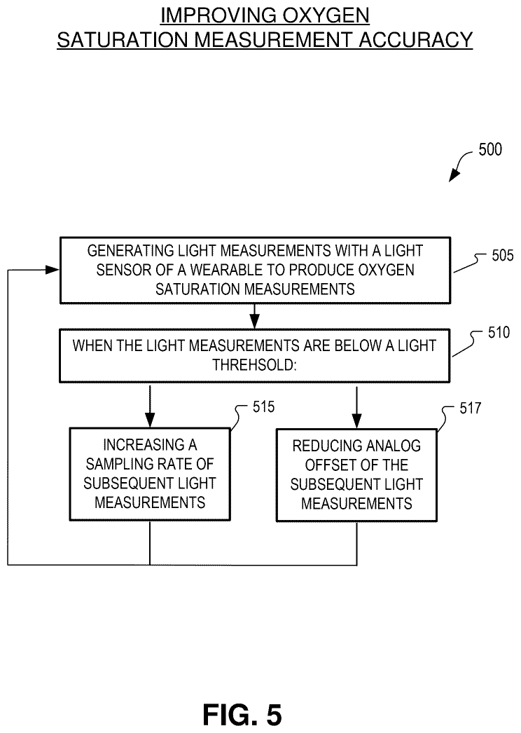

FIG. 5 illustrates a flow chart of an example process 500 of improving oxygen saturation monitoring accuracy, in accordance with aspects of the disclosure. The order in which some or all of the process blocks appear in process 500 should not be deemed limiting. Rather, one of ordinary skill in the art having the benefit of the present disclosure will understand that some of the process blocks may be executed in a variety of orders not illustrated, or even in parallel. All or a portion of the process blocks in process 500 may be executed by processing logic 130 or 430, in some implementations

In process block 505, light measurements are generated with a light sensor of a wearable to produce oxygen saturation measurements. When the light measurements are below a light threshold (e.g. light threshold 141), process 500 executes both process block 515 and 517. In some implementations, only one of process block 515 and 517 is executed after process block 510.

In process block 515, a sampling rate of subsequent light measurements is increased. In some implementations, increasing the sampling rate of the subsequent light measurements includes increasing to an increased sampling frequency of driving the light source and increasing the measuring of the returning light with the light sensor to the increased sampling frequency.

In process block 517, an analog offset of the subsequent light measurements is reduced. In some implementations, reducing the analog offset of the subsequent light measurements includes reducing an offset current (e.g. current 473) that offsets a photodiode current generated by the photodiode during the light measurements.

In some implementations, after executing process block 515 and 517, process 500 may return to process block 505 where the subsequent light measurements are generated at an increased sampling rate and at a reduced analog offset. The light source (e.g. light source 110 or 310) may be driven at a same current for the generating the light measurements and for generating the subsequent light measurements.

In some implementations of process 500, the light measurements are for photoplethysmography (PPG) analysis.

In some implementations of process 500, when the light measurements are not below the light threshold: (1) the subsequent light measurements are generated at the same sampling rate as the initial light measurements generated in process block 505; and (2) the analog offset of the subsequent light measurements is maintained at the same level as the initial light measurements generated in process block 505.

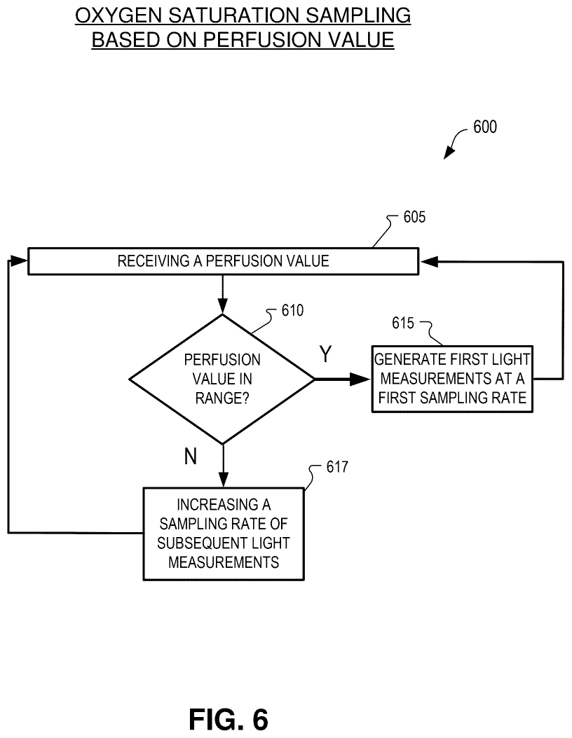

FIG. 6 illustrates a flow chart of an example process 600 of oxygen saturation sampling based on a skin perfusion value, in accordance with aspects of the disclosure. The order in which some or all of the process blocks appear in process 600 should not be deemed limiting. Rather, one of ordinary skill in the art having the benefit of the present disclosure will understand that some of the process blocks may be executed in a variety of orders not illustrated, or even in parallel. All or a portion of the process blocks in process 600 may be executed by processing logic 130 or 430, in some implementations.

In process block 605, a skin perfusion value is received. The skin perfusion value 144 may be received from memory 140, for example.

In decision block 610, the range of the skin perfusion value is determined.

If the skin perfusion value is within a pre-determined value range (e.g. pre-determined value range 145), first light measurements are generated with a light sensor of a wearable at a first sampling rate, in process block 615.

If the skin perfusion value is outside a pre-determined value range (e.g. pre-determined value range 145), second light measurements are generated with a light sensor of the wearable at a second sampling rate, in process block 617.

In an implementation of process 600, the first light measurements are measured at a first analog offset and the second light measurements are measured at a second analog offset that is reduced from the first analog offset.

In an implementation of process 600, generating the first light measurements with the light sensor includes: (1) driving a light source to emit illumination light; and (2) measuring returning light with the light sensor. Generating the first light measurements at the first sampling rate may include having a first sampling frequency of driving the light source and measuring the returning light with the light sensor at the first sampling frequency. Generating the second light measurements at the second sampling rate may include having a second sampling frequency of driving the light source and measuring the returning light with the light sensor at the second sampling frequency that is different from the first sampling frequency.

In an implementation, perfusion data 146 is stored in memory 140 and perfusion data 146 may include a sampling rate of the light measurements and/or a DAC offset value associated with the pre-determined value range 145.

Embodiments of the invention may include or be implemented in conjunction with an artificial reality system. Artificial reality is a form of reality that has been adjusted in some manner before presentation to a user, which may include, e.g., a virtual reality (VR), an augmented reality (AR), a mixed reality (MR), a hybrid reality, or some combination and/or derivatives thereof. Artificial reality content may include completely generated content or generated content combined with captured (e.g., real-world) content. The artificial reality content may include video, audio, haptic feedback, or some combination thereof, and any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to, e.g., create content in an artificial reality and/or are otherwise used in (e.g., perform activities in) an artificial reality. The artificial reality system that provides the artificial reality content may be implemented on various platforms, including a head-mounted display (HMD) connected to a host computer system, a standalone HMD, a mobile device or computing system, or any other hardware platform capable of providing artificial reality content to one or more viewers.

The term “processing logic” (e.g. processing logic 130 or processing logic 430) in this disclosure may include one or more processors, microprocessors, multi-core processors, Application-specific integrated circuits (ASIC), and/or Field Programmable Gate Arrays (FPGAs) to execute operations disclosed herein. In some embodiments, memories (not illustrated) are integrated into the processing logic to store instructions to execute operations and/or store data. Processing logic may also include analog or digital circuitry to perform the operations in accordance with embodiments of the disclosure.

A “memory” or “memories” (e.g. memory 140) described in this disclosure may include one or more volatile or non-volatile memory architectures. The “memory” or “memories” may be removable and non-removable media implemented in any method or technology for storage of information such as computer-readable instructions, data structures, program modules, or other data. Example memory technologies may include RAM, ROM, EEPROM, flash memory, CD-ROM, digital versatile disks (DVD), high-definition multimedia/data storage disks, or other optical storage, magnetic cassettes, magnetic tape, magnetic disk storage or other magnetic storage devices, or any other non-transmission medium that can be used to store information for access by a computing device.

Network 180 may include any network or network system such as, but not limited to, the following: a peer-to-peer network; a Local Area Network (LAN); a Wide Area Network (WAN); a public network, such as the Internet; a private network; a cellular network; a wireless network; a wired network; a wireless and wired combination network; and a satellite network.

Communication channels may include or be routed through one or more wired or wireless communication utilizing IEEE 802.11 protocols, short-range wireless protocols, SPI (Serial Peripheral Interface), I²C (Inter-Integrated Circuit), USB (Universal Serial Port), CAN (Controller Area Network), cellular data protocols (e.g. 3G, 4G, LTE, 5G), optical communication networks, Internet Service Providers (ISPs), a peer-to-peer network, a Local Area Network (LAN), a Wide Area Network (WAN), a public network (e.g. “the Internet”), a private network, a satellite network, or otherwise.

A computing device may include a desktop computer, a laptop computer, a tablet, a phablet, a smartphone, a feature phone, a server computer, or otherwise. A server computer may be located remotely in a data center or be stored locally.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible non-transitory machine-readable storage medium includes any mechanism that provides (i.e., stores) information in a form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A wearable device comprising:
a light source configured to emit illumination light;
a light sensor configured to generate light measurements of returning light, wherein the returning light is a portion of the illumination light; and
processing logic configured to:
generating the light measurements with the light sensor to produce oxygen saturation measurements; and
when the light measurements are below a light threshold: (1) increasing a sampling rate of subsequent light measurements by the light sensor; and (2) reducing an analog offset of the subsequent light measurements.

2. The wearable device of claim 1, wherein generating the light measurements with the light sensor includes: (1) driving the light source to emit the illumination light; and (2) measuring the returning light with the light sensor.

3. The wearable device of claim 2, wherein increasing the sampling rate of the subsequent light measurements includes increasing to an increased sampling frequency of driving the light source and increasing the measuring of the returning light with the light sensor to the increased sampling frequency.

4. The wearable device of claim 3, wherein the light source is driven at a same current for generating the light measurements and for generating the subsequent light measurements.

5. The wearable device of claim 1, wherein the light sensor includes a photodiode, and wherein reducing the analog offset of the subsequent light measurements includes reducing an offset current that offsets a photodiode current generated by the photodiode during the subsequent light measurements.

6. The wearable device of claim 5, wherein the offset current is generated by a digital-to-analog converter (DAC) driven by processing logic, wherein the processing logic is configured to receive an adjusted signal that is a difference between the photodiode current and the offset current, the adjusted signal being measured as part of the subsequent light measurements.

7. The wearable device of claim 1, wherein the processing logic is further configured to:
when the light measurements are not below the light threshold:
generating the subsequent light measurements at the sampling rate; and
maintaining the analog offset for the subsequent light measurements.

8. A method comprising:
generating light measurements with a light sensor of a wearable to produce oxygen saturation measurements; and
when the light measurements are below a light threshold:
increasing a sampling rate of subsequent light measurements for generating subsequent oxygen saturation measurements; and
reducing an analog offset of the subsequent light measurements.

9. The method of claim 8, wherein generating the light measurements with the light sensor includes: (1) driving a light source to emit illumination light; and (2) measuring returning light with the light sensor, wherein the returning light is a portion of the illumination light.

10. The method of claim 9, wherein increasing the sampling rate of the subsequent light measurements includes increasing to an increased sampling frequency of driving the light source and increasing the measuring of the returning light with the light sensor to the increased sampling frequency.

11. The method of claim 10, wherein the light source is driven at a same current for the generating the light measurements and for generating the subsequent light measurements.

12. The method of claim 8, wherein the light sensor includes a photodiode, and wherein reducing the analog offset of the subsequent light measurements includes reducing an offset current that offsets a photodiode current generated by the photodiode during the light measurements.

13. The method of claim 12, wherein the offset current is generated by a digital-to-analog converter (DAC) driven by processing logic, wherein the processing logic is configured to receive an adjusted signal that is a difference between the photodiode current and the offset current, the adjusted signal being measured as part of the subsequent light measurements.

14. The method of claim 8 further comprising:
when the light measurements are not below the light threshold:
generating the subsequent light measurements at the sampling rate; and
maintaining the analog offset of the subsequent light measurements.

15. The method of claim 8, wherein the light measurements are for photoplethysmography (PPG) analysis.

16. A method comprising:
receiving a skin perfusion value;
generating first light measurements with a light sensor of a wearable at a first sampling rate when the skin perfusion value is within a pre-determined value range; and
generating second light measurements with the light sensor of the wearable at a second sampling rate when the skin perfusion value is outside the pre-determined value range.

17. The method of claim 16, wherein the first light measurements are measured at a first analog offset, and wherein the second light measurements are measured at a second analog offset that is reduced from the first analog offset.

18. The method of claim 16, wherein generating the first light measurements with the light sensor includes: (1) driving a light source to emit illumination light; and (2) measuring returning light with the light sensor, wherein the returning light is a portion of the illumination light.

19. The method of claim 18, wherein generating the first light measurements at the first sampling rate includes having a first sampling frequency of driving the light source and measuring the returning light with the light sensor at the first sampling frequency,
and wherein generating the second light measurements at the second sampling rate includes having a second sampling frequency of driving the light source and measuring the returning light with the light sensor at the second sampling frequency different from the first sampling frequency.

20. The method of claim 16, wherein the skin perfusion value is received from a memory of the wearable and perfusion data includes a sampling rate of the light measurements and an offset value associated with the predetermined value range.
